(19) United States
(12) Patent Application Publication
(10) Pub. No.: US 2025/0025714 A1
(43) Pub. Date: Jan. 23, 2025

(54) NON-INVASIVE METHOD FOR SUPPRESSING SPREADING DEPOLARIZATION IN HUMAN BRAINS

(71) Applicant: Carnegie Mellon University, Pittsburgh, PA (US)

(72) Inventors: Alireza Chaman Zar, Pittsburgh, PA (US); Pulkit Grover, Pittsburgh, PA (US); Shawn K. Kelly, Pittsburgh, PA (US); Maysam Chamanzar, Pittsburgh, PA (US); Shilpa George, Pittsburgh, PA (US); Anjali Menon, Pittsburgh, PA (US)

(21) Appl. No.: 18/894,976

(22) Filed: Sep. 24, 2024

Related U.S. Application Data
(63) Continuation-in-part of application No. 17/082,251, filed on Oct. 28, 2020.
(60) Provisional application No. 62/973,839, filed on Oct. 28, 2019.

(51) Int. Cl. A61N 1/40; A61N 1/36; A61N 7/00

(52) U.S. Cl. CPC A61N 1/40; A61N 1/36025; A61N 7/00; A61N 2007/0026

(57) ABSTRACT

Methods providing reliable and non-invasive ways to suppress spreading depolarization waves leading to cortical spreading depression are disclosed. A stimulation signal is applied at the location of and surrounding the location of the spreading depolarization and the amplitude and focal point of the stimulation signal are randomly varied, causing suppression of the spreading depolarization wave.

### RELATED APPLICATIONS

This application is a continuation-in-part of co-pending U.S. patent application Ser. No. 17/082,251, filed Oct. 28, 2020, which claims the benefit of U.S. Provisional Patent Application Ser. No. 62/973,697, filed Oct. 28, 2019, the contents of which are incorporated herein in their entireties.

### GOVERNMENT RIGHTS

This invention was made with government support under contract CNS1702694, issued by the National Science Foundation. The government has certain rights in this invention.

### BACKGROUND OF THE INVENTION

Migraines afflict 3.7 million Americans, and more than 1 billion individuals worldwide. Moreover, every year more than 3 million Acquired Brain Injuries (ABIs), which include traumatic brain injuries (TBIs), strokes, and hemorrhages, happen in the United States. These injuries are the leading cause of deaths and disabilities worldwide.

Cortical Spreading Depression (CSD) are waves of suppression of normal brain activity which propagate across the cerebral cortical surface. The slowly traveling wave (mm/min) is characterized by neuronal depolarization and redistribution of ions between the intracellular and extracellular space, that temporarily depresses electrical activity. This phenomenon is referred to as Cortical Spreading Depolarization (CSD) and occurs in many neurological conditions, such as migraine with aura, ischemic stroke, traumatic brain injury and possibly epilepsy.

In the wake of the propagation of the CSD wavefront, a complex dynamic is triggered. At first, neurons undergo a brief period of intense activity, exhibiting a firing rate 10-20 times higher than when at rest. This brief period of intense excitation is followed by a membrane hyperpolarization which silences the spiking activity for a variable period, after which the neurons slowly recover their spiking activity and eventually return to normal spiking frequency.

CSD is characterized by relevant increases in both extracellular K⁺ and glutamate, as well as rises in intracellular Na⁺ and Ca⁺⁺. The two most accepted hypotheses suggest that propagation of the wave is due to diffusion of potassium or glutamate in the extracellular space.

CSD has been shown to be responsible for worsening brain injuries, and can cause secondary brain damage after TBI, stroke, hemorrhages, etc. Commonly used techniques for CSD suppression involve invasive and/or pharmacological methods. However, due to the side effects of surgery and chemical injections in the brain, and the time it takes to stop CSD using these methods, new methods to suppress CSD are needed. Therefore, finding a reliable, non-invasive way to suppress the CSD is vital.

### SUMMARY OF THE INVENTION

CSD is modelled herein using the standard Tuckwell model, the results of which are consistent with experimental findings about CSD. Using this 2D model, CSD was successfully suppressed by changing the calcium conductance term of the model. In three separate embodiments, the calcium conductance terms was changed in different ways, and, in all cases, the model shows that the CSD was successfully suppressed.

In real brains, the stimulation of focused locations of the brain (i.e. at and near the site of the CSD) with a random stimulation pattern can fully stop CSD waves in as soon as 1 min following onset of the stimulation. Any stimulation technique that can randomly change the membrane potentials in the brain within certain specifications disclosed herein (i.e., having at least 1 cm focality, with the ability to change the stimulation pattern and focal point as fast as 130 ms) can randomly change the calcium conductance and results in complete suppression of the CSD.

### BRIEF DESCRIPTION OF THE DRAWINGS

FIG. 1 are graphs showing the concentrations of the 6 components of the Tuckwell model at time steps t={1, 1000, 1500} of the simulation.

FIG. 2 is a graph showing membrane potential during CSD wave propagation at time step t=1000.

FIG. 3 are graphs showing the extracellular K⁺ concentration (mM) at time steps t={0, 500, 800, 1200} for an embodiment of the invention using Anderson Localization.

FIG. 4 are graphs showing the extracellular concentrations (mM) of the 6 components of the Tuckwell model in one dimension (along the x-axis) at time step t=700.

FIG. 5 are graphs showing the extracellular concentrations (mM) of the 6 components of the Tuckwell model over time at x=y=90.

FIG. 6 shows a pattern for calcium conductance when an aspect of the invention providing localized stimulation is used.

FIG. 7 is a flowchart showing the method.

### DETAILED DESCRIPTION

The Tuckwell mathematical models of CSD wave propagation are considered in the form of reaction-diffusion systems in two space dimensions. CSD consists of local “reaction” processes, such as a release of potassium and glutamate, pump activity and recovery of the tissue in a later stage, as well as diffusion of potassium and glutamate, which enables the propagation of CSD.

The Tuckwell model of CSD, with some modification, is used herein to evaluate the effectiveness of the invention in stopping CSD. The model is based on extracellular (ECS) and intracellular (ICS) ion concentration changes during the CSD propagation. In other words, the model is based on reaction-diffusion of different ions and transmitters. Reaction is referred to the ion exchange between ICS and ECS, and diffusion is referred to the ionic propagation in the ECS.

In the Tuckwell model, 6 components are considered: 4 ions (K⁺, Ca⁺⁺, Na⁺, Cl⁻) and two neurotransmitters, one inhibitory, TI, such as GABA, and one excitatory, TE, which is mainly glutamate. Based on the ECS and ICS concentration of these components, the model comprises 6 coupled 2D parabolic partial differential equations (PDEs) as below:

$$\frac{\partial u}{\partial t} = {∇}^{2} u + F ( u ) \qquad ( 1 )$$

where:
u(x, y, t) is the vector of ECS concentrations with u₁, u₂, . . . , u₆ with the same order as K⁺, Ca⁺⁺, Na⁺, Cl⁻, TE and TI.

The initial condition for these concentrations are defined to put the neurons at rest condition. The initial values and constants used in this model, are:

$$u ( x , y , 0 ) = {u}_{0} ( x , y ) \qquad ( 2 )$$

The boundary conditions were fixed at the initial concentrations. Because the propagation in these simulations starts at the middle of the 2D plane, this assumption seems to be reasonable.

Some assumptions in the CSD model were made. First, the effect of action potentials is ignored for simplicity. This assumption is valid for CSD modeling as there is some evidence to show that even in the absence of action potentials, CSD can still propagate. For example, in case of TTX treatment, which suppresses action potential firing, CSD can still propagate. Second, flows through other membranes are neglected to maintain a degree of simplicity. In addition, it is known that there is a relationship between the dynamics of calcium at the presynaptic region and the amount of transmitter release to the synaptic space, so a major component of calcium fluxes are included in this model.

For the ICS concentrations, there is another set of 4 unknown parameters (there is no TI or TE at ICS) and these parameters will be updated based on the difference between the current value of ECS concentrations and their resting equilibrium values, which are indicated by R. For intracellular concentrations of K⁺, Na⁺, Cl⁻, the following equation is used:

$${u}_{i}^{int} ( x , y , t ) = {u}_{i}^{int . R} + {α}_{1} [ {u}_{i}^{R} - {u}_{i} ( x , y , t ) ] , i = 1 , 3 , 4 \qquad ( 3 )$$

and for ICS Ca⁺⁺ concentration:

$${u}_{2}^{int} ( x , y , t ) = {u}_{2}^{int . R} + {α}_{2} [ {u}_{2}^{R} - {u}_{2} ( x , y , t ) ] \qquad ( 4 )$$

In the model, the membrane potential can be computed using the Goldman formula:

$${V}_{M} = \frac{RT}{F} ln \frac{{K}^{o} + {p}_{Na} {Na}^{o} + {p}_{Cl} {Cl}^{i}}{{K}^{i} + {p}_{Na} {Na}^{i} + {p}_{Cl} {Cl}^{o}} \qquad ( 5 )$$

In the same way, the Nernst potential for all of 4 ions can be computed as below:

$${V}_{K} = \frac{RT}{zF} ln \frac{{K}^{o}}{{K}^{i}} \qquad ( 6 )$$

where z is the ion charge.

In Eq. (1), the F(⋅) function is the flux term for each component which consists of two main parts, active pump current and passive fluxes through ion channels. For K⁺ this term can be calculated as below:

$${f}_{K} = {k}_{1} ( {V}_{M} - {V}_{K} ) [ \frac{{T}_{E}^{o} S ( {T}_{E}^{o} )}{{T}_{E}^{o} + {k}_{2}} + \frac{{k}_{3} {T}_{I}^{o} S ( {T}_{I}^{o} )}{{T}_{I}^{o} + {k}_{4}} ] + {f}_{K , p} + ( {k}_{5} - {P}_{K} ) \vert ( {K}^{o} \neq {K}^{o , R} ) \qquad ( 7 )$$

where, Pₖ is the potassium active pump term and the last term is to make sure that this pump is only active when the concentrations are not at rest values.

In addition, f_{K,p} is the potassium passive flux term as below:

$${f}_{K , p} = {k}_{6} ( {V}_{M} - {V}_{M . R} ) ( {V}_{M} - {V}_{K} ) S ( {V}_{M} - {V}_{M . R} ) \qquad ( 8 )$$

where the S(⋅) function is a step function and V_{M.R} is the resting membrane potential.

There are two separate pump equations for sodium and potassium. These two exchange pump equations can be combined. Here, for potassium:

$$\\ \begin{matrix}{P}_{K} = \frac{{k}_{17} {K}^{{}_{} o} {Na}^{{}_{} i} S ( {Na}^{{}_{} i} ) S ( {K}^{{}_{} o} )}{{K}^{{}_{} o} {Na}^{{}_{} i} + {k}_{18} {K}^{{}_{} o} + {k}_{19} {Na}^{{}_{} i}} & ( 9 )\end{matrix}$$

and for sodium:

$$\\ \begin{matrix}{P}_{K} = \frac{{k}_{22} {K}^{{}_{} o} {Na}^{{}_{} i} S ( {Na}^{{}_{} i} ) S ( {K}^{{}_{} o} )}{{K}^{{}_{} o} {Na}^{{}_{} i} + {k}_{23} {K}^{{}_{} o} + {k}_{24} {Na}^{{}_{} i}} & ( 10 )\end{matrix}$$

For calcium, the F(⋅) function in Eq. (1), is defined as:

$$\\ \begin{matrix}{f}_{Ca} = {k}_{7} ( {V}_{M} - {V}_{Ca} ) {g}_{Ca} + ( {P}_{Ca} - {k}_{8} ) * 1 ( {Ca}^{{}_{} o} \neq {Ca}^{{}_{} o , R} ) & ( 11 )\end{matrix}$$

where the calcium conductance is defined as:

$$\\ \begin{matrix}{g}_{Ca} = ( 1 + tanh [ {k}_{31} ( {V}_{M} + {V}_{M}^{{}_{} *} ) ] - {k}_{32} ) S ( {V}_{M} - {V}_{M}^{{}_{} T} ) & ( 12 )\end{matrix}$$

where, V_{M}^{T} is a cut-off potential and k₃₂ is defined as below to ensure a smooth rise of g_{Ca} from zero:

$$\\ \begin{matrix}{k}_{32} = 1 + tanh [ {k}_{31} ( {V}_{M} + {V}_{M}^{{}_{} *} ) ] & ( 13 )\end{matrix}$$

In addition, calcium active pump can be defined as below:

$$\\ \begin{matrix}{P}_{Ca} = \frac{{k}_{20} {Ca}^{{}_{} i} S ( {Ca}^{{}_{} i} )}{{Ca}^{{}_{} i} + {k}_{21}} & ( 14 )\end{matrix}$$

Similar equations exist for sodium and chloride ion fluxes:

$$\\ \begin{matrix}{f}_{Na} = {k}_{9} ( {V}_{M} - {V}_{Na} ) [ \frac{{T}_{E}^{{}_{} o} S ( {T}_{E}^{{}_{} o} )}{{T}_{E}^{{}_{} o} + {k}_{2}} + \frac{{k}_{10} {T}_{I}^{{}_{} o} S ( {T}_{I}^{{}_{} o} )}{{T}_{I}^{{}_{} o} + {k}_{4}} ] + ( {P}_{Na} - {k}_{11} ) \vert ( {Na}^{{}_{} o} \neq {Na}^{{}_{} o , R} ) & ( 15 )\end{matrix}$$

$$\\ \begin{matrix}{f}_{Cl} = {k}_{12} ( {V}_{M} - {V}_{Cl} ) [ \frac{{k}_{13} {T}_{E}^{{}_{} o} S ( {T}_{E}^{{}_{} o} )}{{T}_{E}^{{}_{} o} + {k}_{2}} + \frac{{T}_{I}^{{}_{} o} S ( {T}_{I}^{{}_{} o} )}{{T}_{I}^{{}_{} o} + {k}_{4}} ] + ( {P}_{Cl} - {k}_{14} ) \vert ( {Cl}^{{}_{} o} \neq {Cl}^{{}_{} o , R} ) & ( 16 )\end{matrix}$$

where the chloride active pump is as below:

$$\\ \begin{matrix}{P}_{Cl} = \frac{{k}_{25} {Cl}^{{}_{} i} S ( {Cl}^{{}_{} i} )}{{Cl}^{{}_{} i} + {k}_{26}} & ( 17 )\end{matrix}$$

Finally, based on the fact that rates of transmitter release are proportional to calcium flux, the transmitter's F(⋅) functions are given as:

$$\\ \begin{matrix}{f}_{TE} = {k}_{15} ( {V}_{m} - {V}_{Ca} ) {g}_{Ca} - {P}_{E} & ( 18 )\end{matrix}$$

$$\\ \begin{matrix}{f}_{TI} = {k}_{16} ( {V}_{m} - {V}_{Ca} ) {g}_{Ca} - {P}_{I} where : & ( 19 )\end{matrix}$$

$$\\ \begin{matrix}{P}_{E} = \frac{{k}_{27} {T}_{E}^{{}_{} o} S ( {T}_{E}^{{}_{} o} )}{{T}_{E}^{{}_{} o} + {k}_{28}} & ( 20 )\end{matrix}$$

$$\\ \begin{matrix}{P}_{I} = \frac{{k}_{29} {T}_{I}^{{}_{} o} S ( {T}_{I}^{{}_{} o} )}{{T}_{I}^{{}_{} o} + {k}_{30}} & ( 21 )\end{matrix}$$

These pump terms work to return transmitters, such as GABA and glutamate, back to the glia cells. As previously stated, in this model, the effect of action potential firing and the consequent change in the ion concentrations are ignored and this can be translated into the fact that the effect of glutamate release from glia cells during CSD have also been ignored.

For purposes of modeling embodiments of the invention, the CSD propagation is instigated by means of a local elevation of ECS potassium concentration using KCL stimulation with a spatial-exponential profile as below:

$$\\ \begin{matrix}{K}^{{}_{} o} ( x , y , 0 ) = {K}^{{}_{} o , R} + 17 exp [ - \{ {( \frac{x - 1}{0.05} )}^{2} + {( \frac{y - 1}{0.05} )}^{2} \} ] & ( 22 )\end{matrix}$$

$$\\ \begin{matrix}{Cl}^{{}_{} o} ( x , y , 0 ) = {Cl}^{{}_{} o , R} + 17 exp [ - \{ {( \frac{x - 1}{0.05} )}^{2} + {( \frac{y - 1}{0.05} )}^{2} \} ] & ( 23 )\end{matrix}$$

To solve these coupled parabolic 2D partial differential equations, Euler's method can be used. The equations should first be discretized in this way:

$$\\ \begin{matrix}\frac{1}{Δ t} ( {U}_{m}^{{}_{} n} - {U}_{m}^{{}_{} n - 1} ) = \frac{D}{2 Δ {x}^{{}_{} 2}} [ {U}_{m + 1}^{{}_{} n - 1} - 2 {U}_{m}^{{}_{} n - 1} ] + F ( \frac{3}{2} {U}_{m}^{{}_{} n - 1} - \frac{1}{2} {U}_{m}^{{}_{} n - 2} ) & ( 24 )\end{matrix}$$

where, D is the scaled diffusion coefficient of the corresponding component

$$\\ ( \times {10}^{{}_{} 2} \frac{{cm}^{2}}{sec} ) .$$

In this solution, two previous time indices, n−1 and n−2, are used to make the updates of the parameters smoother. In this simulation, the spatial unit step is equal to 5.2 mm and the temporal unit step is equal to about 26 sec. The dimension of the 2D plane in this simulation is a 2×2 spatial unit and the total duration of simulation is 10 temporal units.

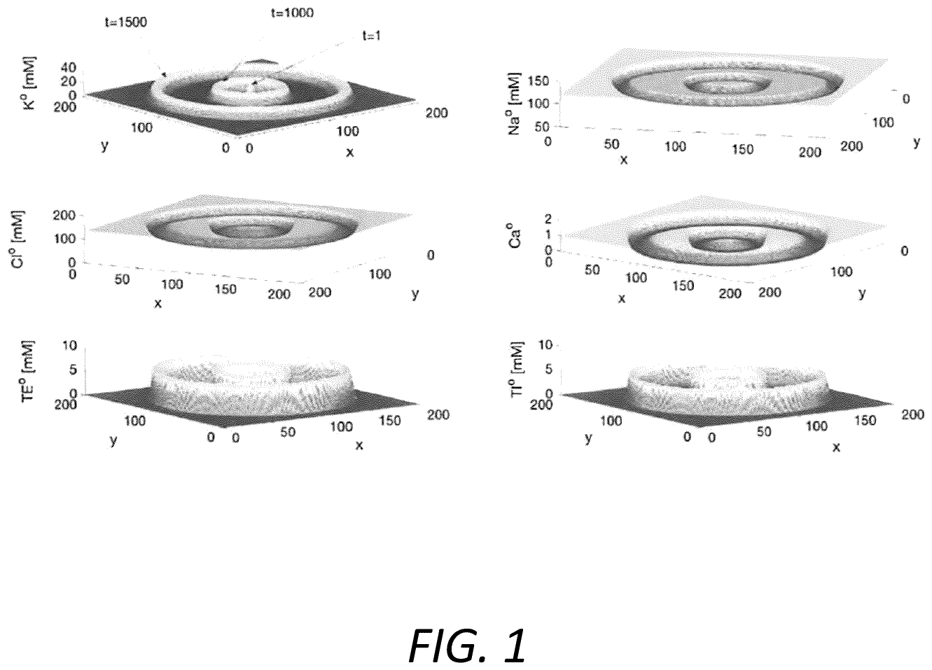

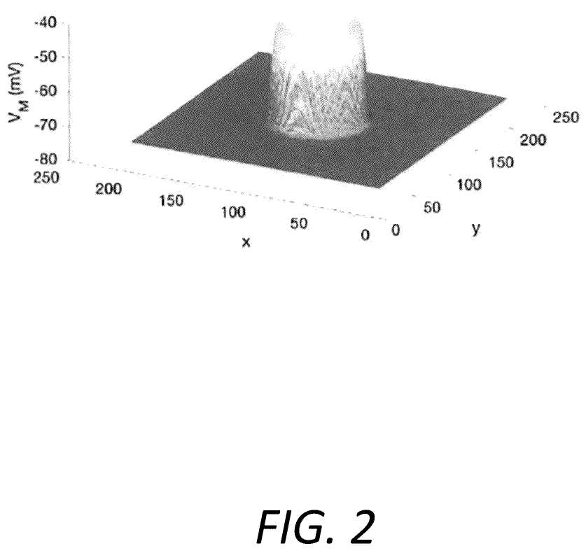

As shown in FIG. 1, the changes in the ECS concentrations of all 6 components of the Tuckwell model are shown at three different time points, which are obtained by means of numerical solution of the above equations. These results are consistent with the experimental findings, but there are still some unknowns. In particular, the physiological mechanism of increasing ECS potassium concentration, other than action potential, during CSD propagation still remains a question. FIG. 2 is an illustration of membrane potential (V_{M}) in 2D. As can be seen, during the CSD propagation, there is a slow increase (because of the slow speed of propagation of CSD) in the membrane potential, and this increase is around the required threshold for action potential firing, but, surprisingly, there is no firing. One possible explanation for this is that because the increase in the amplitude of membrane potential is gradual, the cell does not fire.

Once the model of CSD propagation has been implemented, the next task at hand is CSD suppression.

In a first embodiment of the invention, the calcium conductance was randomized by applying an Anderson Localization. Anderson localization is a general concept that may be applied to any wave system. It entails the randomization of the medium through which the wave is travelling and is used in this invention to disrupt the CSD wave. In case of CSD, this is randomization of the cortical medium. Parameters related to firing rates and threshold potentials were randomized to achieve Anderson localization.

In the Tuckwell model, the calcium conductance term g_{Ca}, depends on the membrane voltage, and, as such, can be randomized using Anderson Localization. The calcium conductance term g_{Ca} in turn controls the flux terms of Ca⁺⁺, Glutamate and GABA in the 6 component Tuckwell model. In this embodiment of the invention, the constant k₃₁ is replaced by a Gaussian random process.

$$\\ \begin{matrix}{k}_{31} \to 𝒩 ( {k}_{31} , {k}_{31}^{{}_{} 2} ) & ( 25 )\end{matrix}$$

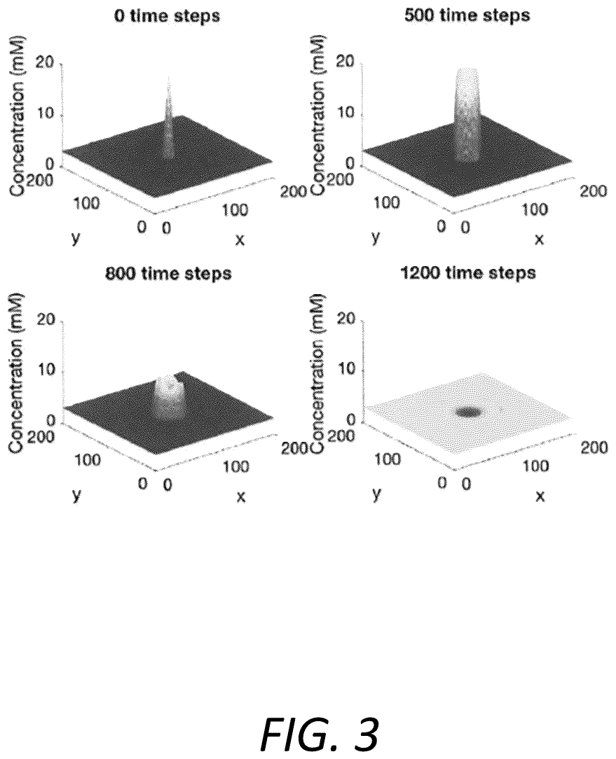

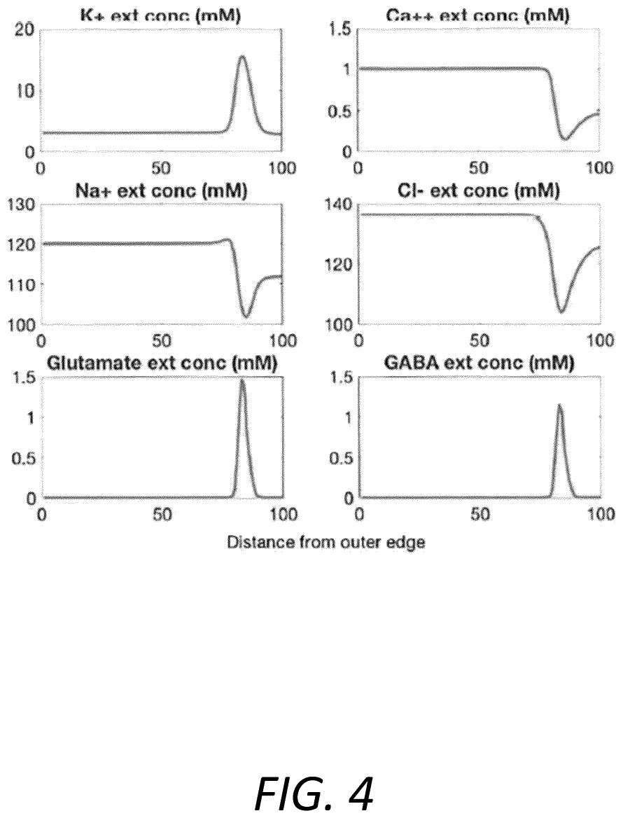

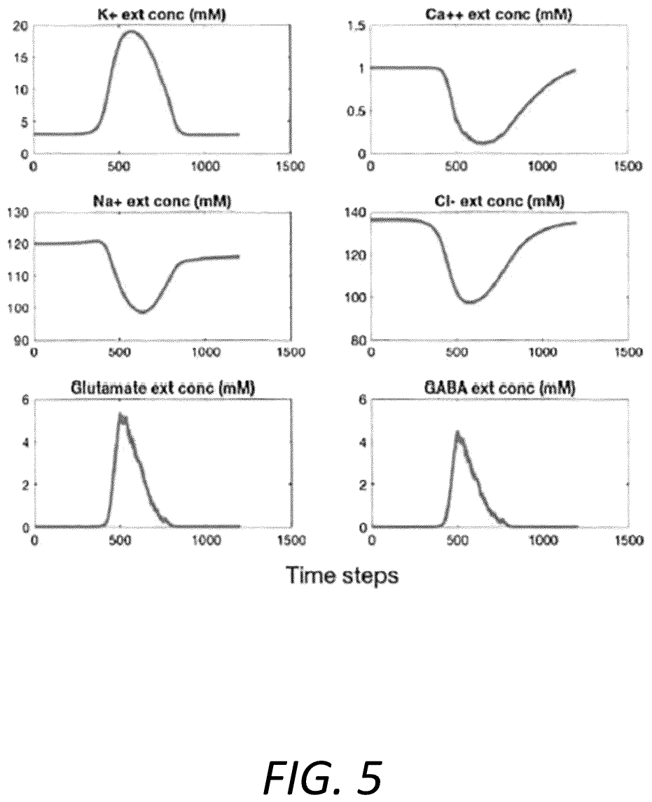

Randomizing the k₃₁ parameter in accordance with the Gaussian function results in CSD suppression. The result of randomizing calcium conductance on extracellular K⁺ concentrations is shown in FIGS. 3-5. The randomization was introduced at t=500 time steps. FIG. 3 clearly shows that the CSD suppression after randomization is introduced. FIG. 4 and FIG. 5 show the extracellular concentrations of all 6 components of the Tuckwell model at a specific instant of time (FIG. 4) and at a specific location (x=y=90, FIG. 5). This embodiment of the invention may be implemented for use on an actual brain using a transcranial random noise stimulation (TRNS) process to randomize the calcium conductance in the intracellular space by randomizing both the frequency and the amplitude of the electrical signal injected into the brain.

In a second embodiment of the invention, the calcium conductance term may be scaled in accordance with a constant.

$$\\ \begin{matrix}{k}_{31} \to {ck}_{31} & ( 26 )\end{matrix}$$

where c is a constant that will either increase of decrease the k₃₁ parameter.

In scenarios as applied to an actual living brain, the technique depends on both the spatio-temporal randomness of the location of the focal point of the stimulation in the brain and the randomness of the stimulation amplitude, to follow a normal random process, regardless of the mechanism of the delivery of the stimulation signal.

A random stimulation pattern with a focality as large as 1 cm³ is used to stimulate a 3D region of the brain (e.g., a sphere-shaped, cube-shaped, toroid shaped area) at and around the site of CSD propagation, which can fully stop CSD waves in as fast as 1 min following the onset of the delivery of the stimulation signal. The stimulation pattern needs to change as fast as 130 ms with a random amplitude following a normal distribution, accompanied by a random change in locality of the focal point of the stimulation, also in accordance with a normal distribution. That is, randomly moving stimulation focal point within a region in the brain, including at the site of CSD propagation and, in preferred embodiments, within 1 cm of the location of the CSD. In preferred embodiments, the stimulation signal is delivered with a random amplitude in a range of 0-2 mA generated following a Gaussian normal random process with zero mean, 1 mA standard deviation, with a frequency in the approximate range of around 8 Hz-1 KHz. A frequency of at least 8 Hz provides a change in the focal point and amplitude as quickly as every 130 ms.

Any stimulation technique that can randomly change the membrane potentials in the brain within the specifications stated above (having at least 1 cm focality, with the ability to change the stimulation pattern and focal point as fast as 130 ms) can randomly change the calcium conductance and results in complete CSD suppression.

Transcranial electrical stimulation techniques such as high density electrical current stimulation (HD-ECS) can provide the focality (as small as 1 mm³) and speed (as fast as 20 ms stimulation time) needed to implement CSD suppression in accordance with this invention. The stimulation pattern is steered in real time without physical movement of the electrodes, using the process described below. It works based on a multi-electrode system that injects currents into the brain with a specific set of parameters (i.e., stimulation waveform, current amplitude, and a choice and placement of a subset of electrodes to use on scalp). Specific parameters can be determined in one embodiment using a machine learning method such as is described in U.S. patent application Ser. No. 18/021,257, entitled “Method for Focused Transcranial Electrical Current Stimulation”, which is incorporated herein by reference and which describes a machine learning method for determining the parameters required to focus the stimulation signal. In other embodiments, an optimization algorithm known as the Directionally Constrained Maximization (DCM) could also be used. These algorithms take the desired location, focality, and intensity as a set of inputs and produce the set of stimulation parameters to be used in a HD-ECS system to stimulate the region of interest. A high density electrode placement on the scalp at the 10-5 standard locations with 256 electrodes along with the HD-ECS optimization techniques can be utilized to achieve the focality needed for CSD suppression. Aside from the electrode locations, which are at standard 10-5 system locations, other parameters such as waveforms, amplitude at each electrode, and choice of electrode subset can also be found using the machine learning and optimization techniques mentioned above. The desired focal point, stimulation intensity (current density at the site of stimulation) and electrode placement are provided as inputs to the machine learning algorithm and the other stimulation parameters needed for achieving the desired focality of the stimulation are returned.

The concept for focusing the stimulation signal is similar to beamforming technology, using constructive patterns of electric fields at the site of stimulation and destructive patterns outside the stimulation focus. Imagine multiple sources are injecting currents into the brain and at some places in the brain they interfere with each other. The described machine learning and optimization methods can design these interferences to achieve the desired focal stimulation.

ECS techniques also can be focused in the brain using temporal interference (TI). TI has high resolution even at depth, and its resolution could be improved even further using symmetric multielectrode placements on scalp. The idea is similar to the machine learning and optimization methods described above, but rather than spatial interference of the stimulation currents in the brain, it is based on the temporal interference of sinusoids in which pairs of electrodes inject sinusoidal currents in the brain at a very high frequency (around 2 kHz) with a very small frequency difference (e.g., 2.01 kHz and 2 kHz, where delta f=10 Hz) between the pair of signals. Neurons don't respond to high frequency sinusoids but rather to the small frequency difference at the location in the brain where these two sinusoids interfere. The superposition of two sinusoids results in a sinusoid with a very slow envelope changing at the rate of delta f (e.g., 10 Hz) which then stimulates the neurons at the site of wave interference.

The location of the CSD in the brain can be determined using any known method. One such method is described in U.S. patent application Ser. No. 18/689,207, entitled “System and Method for the Non-Invasive Detection of Spreading Depolarization Using EEG”, which is incorporated herein by reference. Once the location of the CSD is known, the locality of the stimulation can be rapidly changed within a volume in the brain, including, in preferred embodiments, at the site of CSD propagation and within approximately 1 cm of the focus of the CSD.

The shape of the waveform depends on the physical implementation of the random stimulation. For example, it can be a short pulse or a DC waveform in a multi-electrode ECS system or even TMS, or a sinusoidal waveform in TI or ultrasound techniques (in which the stimulation signal is ultrasonic and is delivered by two or more transducers). What matters here is the spatio-temporal randomness of the stimulation focal point in the brain and the randomness of the magnitude of the stimulation amplitude following a normal random process, with a minimum frequency of around 8 Hz (resulting in change in the focal point and amplitude in as fast as 130 ms).

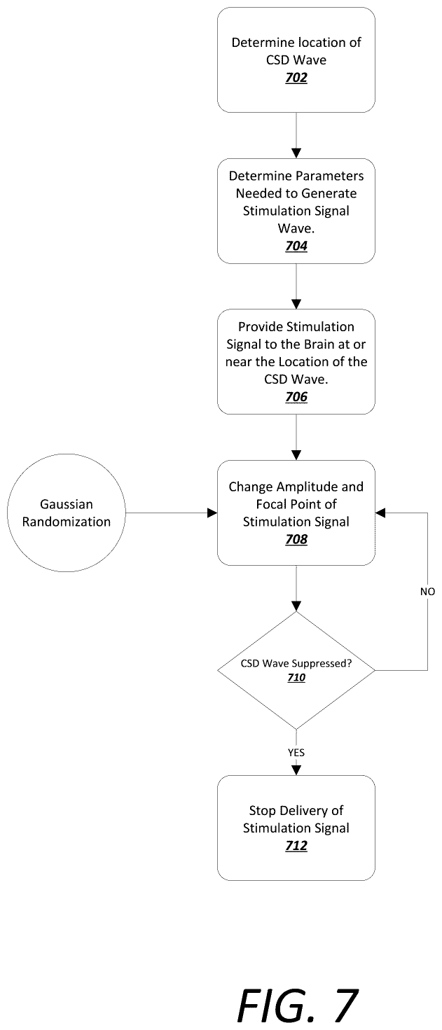

FIG. 7 is a flowchart showing the steps of the claimed method of one embodiment of the invention. At 702, the location of the CSD wavefront is detected, using, for example, the method set forth in previously-discussed U.S. patent application Ser. No. 18/689,207. At 704, the parameters required to generate the stimulation signal are determined, using one of the previously-discussed machine learning or optimization methods. AT 704, the stimulation signal is applied to the brain, using one of the techniques and hardware methods previously discussed. At 708, the amplitude and focality of the stimulation signal are changed in accordance with a Gaussian process. At 710, it is determined if the CSD wave gas been suppressed, and, if not, the amplitude and focality characteristics of the stimulation signal are further varied. If the CSD wave is successfully stopped at 710, at 712, the delivery of the simulation signal is stopped.

In real life scenarios, this embodiment may be implemented using a transcranial direct current stimulation (TDCS) process to scale the calcium conductance in the intracellular space. In a separate aspect of this embodiment, the value of c may be varied around a mean by randomly changing the amplitude of the TDCS signal.

In yet a third embodiment of the invention, the calcium conductance term may be changed in accordance with a spatial sinusoidal wave function in which may be implemented using a transcranial alternating current stimulation (TACS) process to change the calcium conductance in the intracellular space in accordance with a deterministic wave.

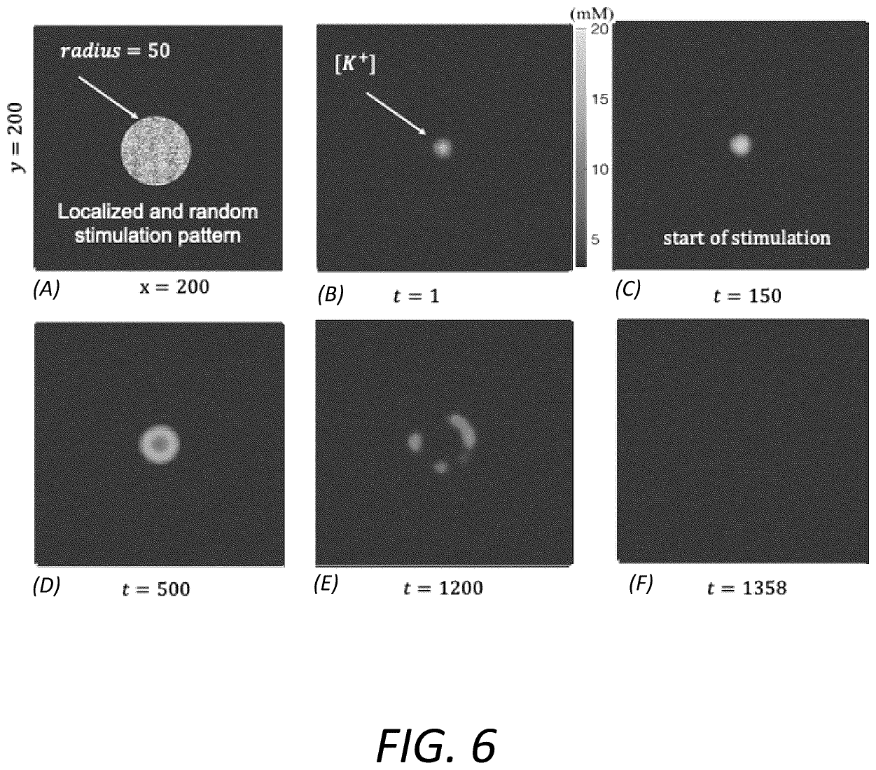

In an aspect of the invention, all three of the described embodiments may be localized to a particular area of the brain experiencing CSD to provide localized change in the calcium conductance as opposed to a global, brain-wide change. Localized randomization of calcium conductance is equally effective to rapidly and completely suppress and inhibit the CSD in the cortical space. FIG. 6 shows this result. FIG. 6(A) shows the spatial pattern of stimulation is shown for k₃₁ (Eq. (12) describes how g_{Ca} changes as a function of k₃₁). This pattern is a localized randomization of k₃₁ inside a disk with a radius of only r=50 (in a 200×200 Tuckwell model), where the center of the disk is aligned with the origin of an instigated CSD (x₀, y₀) in this model:

$$\\ \begin{matrix}{k}_{31} = \{ \begin{matrix}𝒩 ( {ck}_{31} , {k}_{31}^{{}_{} 2} ) , c > 1 , & {( x - {x}_{0} )}^{2} + {( y - {y}_{0} )}^{2} \leq {r}^{{}_{} 2} and t \geq {t}_{0} \\ {k}_{31} , & {( x - {x}_{0} )}^{2} + {( y - {y}_{0} )}^{2} \geq {r}^{{}_{} 2} or t \leq {t}_{0}\end{matrix} & ( 27 )\end{matrix}$$

This stimulation pattern is applied at t₀=150, as shown in FIG. 6(C), and continues until the full suppression of CSD at t=1358, shown in FIG. 6(F). Such localized stimulation patterns may be achieved in practice using current stimulation methods such as temporal interference (TI).

In another aspect of the embodiments of the invention, a phased array of ultrasound transducers may be used to provide neuromodulation.

Neuromodulation using ultrasound has also been seen to stimulate Na⁺ and Ca⁺⁺ channel activity leading to an increased firing of neurons. These may have a role to play in neurotransmitter-based ion channels. Ultrasound has the advantage of being non-invasive, although incurring the cost of poor spatial resolution. This kind of neuromodulation is known to affect membrane permeability which, in turn, would have an influence on conductance. In the Tuckwell model, only calcium conductance is assumed to play a significant role towards CSD propagation and conductance associated with K⁺ and Na⁺ have been ignored. Using ultrasonic neuromodulation, it is possible to randomize calcium conductance to achieve the simulated result shown in FIG. 3.

Cortical spreading depression caused by CSD has been implicated in migraine and as a headache trigger and, in the case of an acutely injured brain, the depolarization waves cause secondary neuronal damage. Understanding the mechanism of CSD is paramount to the objective of suppressing these waves. It is clear that a large number of neural, glial, synaptic, metabolic and neurochemical variables are involved in some way with the formation and passage of a CSD wave.

This invention has introduced several methods of suppressing the CSD waves by modifying concentrations of the ions and neurotransmitters important to the sustenance of CSD. In some embodiments, the invention described herein includes methods that can vary neural parameters, particularly, but not limited to, calcium conductance across space and time, to suppress CSD waves.

1. A method of suppressing a spreading depolarization in a brain comprising:
determining a location of the spreading depolarization in the brain;
providing a stimulation signal to the brain focused on a volume of the brain at and surrounding the determined location of the spreading depolarization; and
randomly varying an amplitude and focal point of the stimulation signal to suppress the a spreading depolarization.

2. The method of claim 1 wherein the amplitude and the focal point of the stimulation signal are varied in accordance with a normal distribution.

3. The method of claim 2 wherein the volume of the brain on which the stimulation is focused is approximately 1 cm³ in volume.

4. The method of claim 1 wherein the amplitude and focal point are changed at a minimum every 130 ms.

5. The method of claim 1 wherein the location of the focal point is varied from the location of the spreading depolarization a maximum of 1 cm.

6. The method of claim 1 wherein the amplitude of the stimulation signal is varied in a range between 0-2 mA.

7. The method of claim 6 wherein the amplitude is varied in accordance with a Gaussian normal random process with 0 mean and 1 mA standard deviation.

8. The method of claim 1 where the focal point is steered using spatial and/or temporal interference techniques.

9. The method of claim 8 wherein the spatial interference technique includes using constructive and destructive interference of two or more sources of the stimulation signal to change the focal point of the stimulation signal.

10. The method of claim 8 wherein the temporal interference technique uses frequency differentials between pairs of sources of the stimulation signal to change the focal point of the stimulation signal.

11. The method of claim 1 wherein the stimulation signal comprises one or more electric fields delivered by one or more electrodes.

12. The method of claim 1 wherein the stimulation signal is delivered by transcranial electrical stimulation.

13. The method of claim 12 wherein the transcranial electrical stimulation is high-density electrical current stimulation.

14. The method of claim 1 wherein the stimulation signal is ultrasonic and is delivered by two or more transducers.

15. The method of claim 1 wherein membrane potentials in the brain are altered by the stimulation signal.

16. The method of claim 1 wherein random variation of the amplitude and focal point of the stimulation signal randomizes the cortical medium through which the spreading depolarization wave passes, thereby suppressing the spreading depolarization.

17. The method of claim 1 wherein the random variation of the amplitude and focal point of the stimulation signal causes an Anderson localization to form in the cortical medium of the brain.

18. The method of claim 17 wherein the Anderson localization causes a randomization of the calcium conductance in the intracellular spaces of the brain.
